(19) United States
(12) Patent Application Publication
(10) Pub. No.: US 2026/0335547 A1
(43) Pub. Date: Oct. 29, 2026

(54) MODULAR INFUSION CONTROL DEVICE AND METHOD

(71) Applicant: CareFusion 303, Inc., San Diego, CA (US)

(72) Inventors: Daniel M. ABAL, San Diego, CA (US); Brendan BURGESS, Poway, CA (US)

(21) Appl. No.: 19/120,177

(22) PCT Filed: Oct. 28, 2022
(86) PCT No.: PCT/US2022/048290

§ 371 Date: Apr. 10, 2025

(51) Int. Cl. A61M 5/172; G16H 20/17

(52) U.S. Cl. CPC A61M 5/1723; G16H 20/17

(57) ABSTRACT

An infusion control module is configure to electrically connect to a patient care unit such as a medication infusion system, receive an indication that the infusion control module was electronically coupled to a main frame of the patient care unit, and to switch the patient care unit to a mode in which the patient care unit is controlled by the control module. The control module determines that the patient care unit is programmed to deliver a medication using an infusion device module coupled to the main frame, determine that the medication is compatible with a therapy previously programmed into the infusion control module, and cause, while electronically coupled to the main frame infusion controller, the infusion device module to deliver the medication according to the therapy.

### BACKGROUND

This application relates generally to control of infusion devices.

Patient care units may include modular infusion platforms that are expandable with multiple medication delivery modules to handle more than one type of medication delivery to a patient. Different patients require different levels of treatment, and different parameters for different devices. Each individual infusion device may operate differently, and include a different type of user interface, resulting in a wide variety of display types and parameter variations that may confuse or distract clinicians during infusion and other medication delivery procedures.

Moreover, modern infusion devices are mobile and able to administer medications to a patient as the patient moves between care areas throughout a medical environment such a hospital organization. Failure to maintain control over the devices in different environments increases the health risk to the patients of a healthcare facility. Furthermore, some infusion devices are not suitable to switch between different medical environments, particularly when critical care may require moving a patient between different care units and different devices quickly.

### SUMMARY

The subject technology provides a device, system and method for intelligently controlling medical devices, and more particularly infusion devices, using a modular infusion control device. An intelligent infusion control module of the subject technology provides direct control over connected infusion devices to assist the clinician in providing a more centralized control and management of the devices. The control module provides a modular interface system attaches to legacy devices and interfaces with external sensors and devices to facilitate control over the connected medical devices. The module is configured to take over control of a medication administration by a pump and then disconnect from the pump and continue control of the medication administration using a different pump. The control module may further connect to server and cloud-based systems for further data input, data coordination, and reporting.

In this regard, the subject technology includes an infusion control module configured to receive an indication that the infusion control module was electronically coupled to a main frame infusion controller, the main frame infusion controller being configured to control one or more infusion device modules coupled to the main frame infusion controller; determine, by the infusion control module, that the main frame infusion controller is programmed to control delivery of a medication using an infusion device module coupled to the main frame infusion controller; determine, by the infusion control module, that the medication is compatible with a therapy previously programmed into the infusion control module; and cause, by the infusion control module while electronically coupled to the main frame infusion controller, the infusion device module to deliver the medication according to the therapy. Other aspects include corresponding devices, methods, and computer program products for implementation of the corresponding system and its features.

A machine-implemented method comprises receiving, by an infusion control module, an indication that the infusion control module was electronically coupled to a main frame infusion controller, the main frame infusion controller being configured to control one or more infusion device modules coupled to the main frame infusion controller; determining, by the infusion control module, that the main frame infusion controller is programmed to deliver a medication using a infusion device module coupled to the main frame infusion controller; determining, by the infusion control module, that the medication is compatible with a therapy previously programmed into the infusion control module; and causing, by the infusion control module while electronically coupled to the main frame infusion controller, the infusion device module to deliver the medication according to the therapy. Other aspects include corresponding devices, systems, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B is a closer view of a portion of the example patient care unit shown in FIG. 1A, according to various aspects of the subject technology.

FIG. 2 illustrates a patient care unit with connected infusion device modules 18, 20 configured to deliver a fluid, a main frame infusion controller, and a modular infusion control device, according to various aspects of the subject technology.

FIG. 3 depicts an example flow diagram for controlling an infusion device using a modular infusion control module, according to aspects of the subject technology.

FIG. 4 depicts an example process for controlling an infusion device using a modular infusion control module, according to aspects of the subject technology.

FIG. 5 is a conceptual diagram illustrating an example electronic system for controlling an infusion device using a modular infusion control module, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology provides an intelligent infusion control module, including a mobile unit that provides processing for control algorithms, and connectivity to enable closed and semi-closed-loop control capabilities over one or medical devices at a point of use. In some implementations, the control module is configured to operably connect to legacy infusion device interfaces to provide operative support and technological expansion to legacy equipment. In this manner, the control module may couple to an infusion pump and take over control of the pump's control systems, providing updated functionality to existing pump systems. While under control the pump's fundamental system for medication administration may continue to operate using legacy circuitry but with monitoring, control and oversight provided by the control module. In this regard, the pump be expanded with technology provided by the control module without further modification to the pump mechanics.

The control module may further provide an external interface between a pump and one or more different physiological sensors, and provide input parameters that are to be used for controlling the titration of IV infusions of medications to a patient. In this regard, the control module can incorporate control software (including, e.g., one or more algorithms) that can be tailored to specific or general medical treatments.

A closed-loop control system generally refers to a system that does not rely on external manual inputs to deliver a therapy. Once configured, the closed-loop system can autonomously provide a therapy, receive feedback from one or more sensors, and, based on the feedback, automatically adjust the therapy as needed. A semi-closed-loop control system is similar to a closed-loop control system except that in some circumstances, the adjustment to the therapy may depend on an external input. In some implementations, a semi-closed-loop control system may be referred to as a decision support system.

According to various implementations, the control module of the subject technology may utilize control software that is separate from the embedded firmware of both the pumps, and from sensors, and which can facilitate a scalable and rapidly configured system to provide closed-loop control of medical treatments. Moreover, the control module can be configured to operate with a multitude of sensors by way of electrical connectors and/or wireless communication. The module may include one or more microprocessors and algorithms to provide signal conditioning and/or conversion of the sensor signals to the appropriate physiological parameters for the connected infusion device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

Having the control module provide control over pumps as a separate unit provides several advantages. For example, the advancement of sensors may be much more rapid than the development of infusion pump systems, and thus the control system may accommodate these changes more quickly. It may be desirable to update control algorithms to address changes in treatment methods, available medications, and patient physiology. For this reason, it may be desirable to have the algorithm reside in the control module rather than the pump system, in order to accommodate more frequent changes. Moreover, machine learning and artificial intelligence may account for patient variations related to physiological parameters such as age, genetics, health history, and other characteristic and environmental factors. Systems incorporating such capabilities may involve large databases and complex programs requiring powerful microprocessors and data storage capabilities to perform the timely and accurate computation needed. Where such systems are generally not capable of running on the systems currently available with the IV pumps alone, the control module may connect to and leverage such systems to facilitate operation of the connected infusion device.

Additionally, the control module of the subject technology is adaptable to a variety of pump systems and sensor inputs. The control module may further be configured to add wireless, Bluetooth and LAN connections to pump systems that do not currently have it available. Adding such communications to the pump system may enable other capabilities such as remote monitoring and control of the infusion pumps, and the access to patient EMR. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

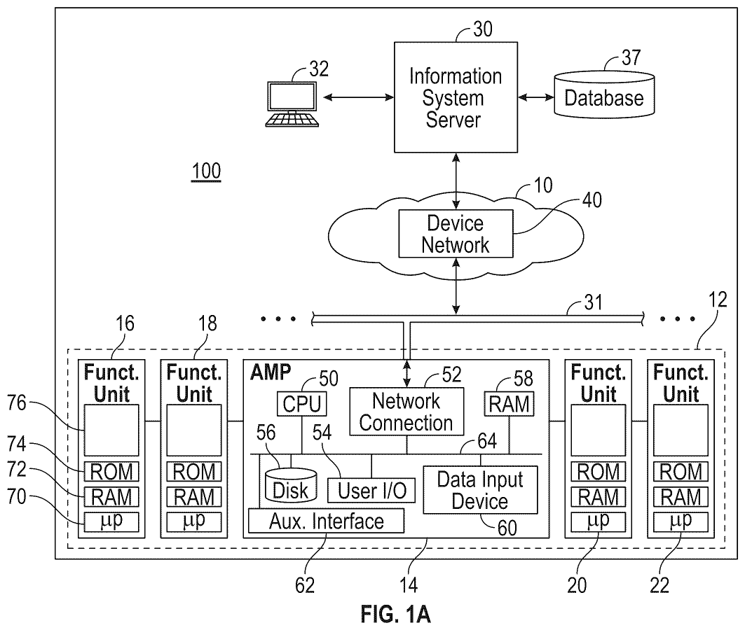

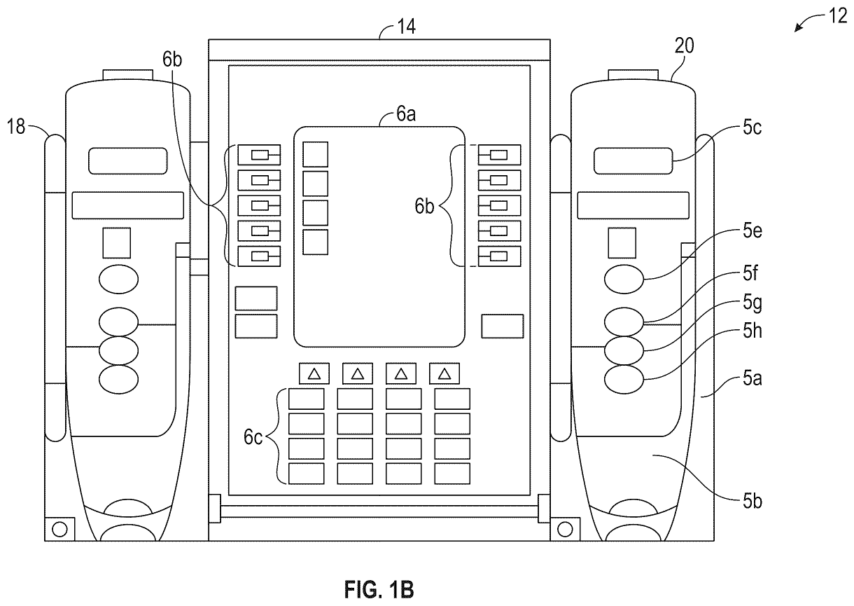

FIG. 1A depicts an example of an institutional patient care system 100 of a healthcare organization, according to aspects of the subject technology. In FIG. 1A, a patient care unit 12 (or “medical device” or “infusion device”, generally) is connected to a hospital network 10. The patient care unit (or “PCU”) may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 502.11 wireless local area network (LAN). In some implementations, network 10 also includes computer systems located in various departments throughout a hospital. For example, network 10 of FIG. 1A optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care units 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care unit 12 comprises a system for providing patient care, such as that described in Eggers et al., which is incorporated herein by reference for that purpose. Patient care unit 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care unit 12 comprises a control module 14, also referred to as main frame infusion controller 14, connected to one or more functional modules 16, 18, 20, 22. Main frame infusion controller 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Main frame infusion controller 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1A to be disposed within main frame infusion controller 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with a main frame infusion (controller) device 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1A, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may be a printer, scanner, bar code reader, near-field communication reader, RFID reader, or any other peripheral input, output or input/output device.

Each functional module 16, 18, 20 and/or 22 communicates directly or indirectly with main frame infusion controller 14, with main frame infusion controller 14 providing overall monitoring and control of device 12. Functional modules 16, 18, 20 and/or 22 may be connected physically and electronically in serial fashion to one or both ends of main frame infusion controller 14 as shown in FIG. 1A, or as detailed in Eggers et al. However, it is recognized that there are other means for connecting functional modules with the main frame infusion controller that may be utilized without departing from the subject technology. It will also be appreciated that devices such as pumps or patient monitoring devices that provide sufficient programmability and connectivity may be capable of operating as stand-alone devices and may communicate directly with the network without connected through a separate main frame infusion controller 14. As described above, additional medical devices or peripheral devices may be connected to patient care unit 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1B, any number of devices may be connected directly or indirectly to main frame infusion controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, main frame infusion controller 14 monitors and controls overall operation of device 12. For example, as will be described in more detail below, main frame infusion controller 14 provides programming instructions to the functional modules 16, 18, 20, 22 and monitors the status of each module.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care unit 12 and network 10 may communicate via automated interaction, manual interaction or a combination of both automated and manual interaction. Automated interaction may be continuous or intermittent and may occur through direct network connection 52 (as shown in FIG. 1A), or through RS232 links, MIB systems, RF links such as BLUETOOTH, IR links, WLANS, digital cable systems, telephone modems or other wired or wireless communication means. Manual interaction between patient care unit 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

According to various implementations, server 30 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information system 30. The pharmacy reviews the order, and once the order has been prepared, the order may be transmitted to the nurse station for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The formulary's establishment of these parameters, along with parameters for off-formulary orders, via the system 30 is useful for maintaining consistency across the healthcare environment and ensuring an order is intelligible and executed according to expectations by other devices within the system 30 (e.g., an infusion pump).

With further reference to FIG. 1A, patient care unit 12 is capable of operating in several different modes, or personalities, with each personality defined by a configuration database. The configuration database may be a database 56 internal to patient care unit, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care unit's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the main frame infusion controller 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the patient care unit 12 (including, e.g., main frame 14 and/or connected modules) and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

FIG. 1B is a closer view of a portion of the example patient care unit 12 shown in FIG. 1A, according to various aspects of the subject technology. FIG. 1B shows two functional infusion pump modules 18, 20 (e.g., “infusion pumps”) mounted at either side of a main frame infusion controller 14, and the displays and control keys of each, with the main frame infusion controller being capable of programming both infusion pumps. The infusion device includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages). Control keys 5e-h may exist for programming and controlling operations of the infusion pump as desired. In some implementations, the control keys may be presented as interactive elements on the display 5c (e.g., touchscreen display). The main frame and/or functional module may also include audio alert equipment in the form of a speaker (not shown).

The main frame infusion controller 14 of the patient care unit 12 includes a display 6a for visually communicating various information, such as the operating parameters of a connected pump and alert indications and alert messages, and control keys 6b and 6c for selecting and/or setting control parameters and/or options for controlling the patient care unit 12 and connected modules. The main frame infusion controller 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The main frame infusion controller 14 may include a communications system (not shown) with which the main frame infusion controller 14 may communicate with external equipment such as a medical facility server or other computer and with a portable processor, such as a handheld communication device or a laptop-type of computer, or other information device that a clinician may have to transfer information as well as to download drug libraries to a functional module 16, 18, 20, 22 (such as pump). The communication module may be used to transfer access and interaction information for clinicians encountering the main frame infusion controller or device coupled therewith (e.g., pump 20 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a main frame infusion controller is not used, or in addition to one with the main frame infusion controller 14. Further, information input devices need not be hard-wired to medical instruments, information may be transferred through a wireless connection as well. Additionally, other types of modules may be connected to the pump modules or to the main frame infusion controller such as a syringe pump module, patient controlled analgesic module, End Tidal CO₂ monitoring module, oximeter monitoring module, or the like.

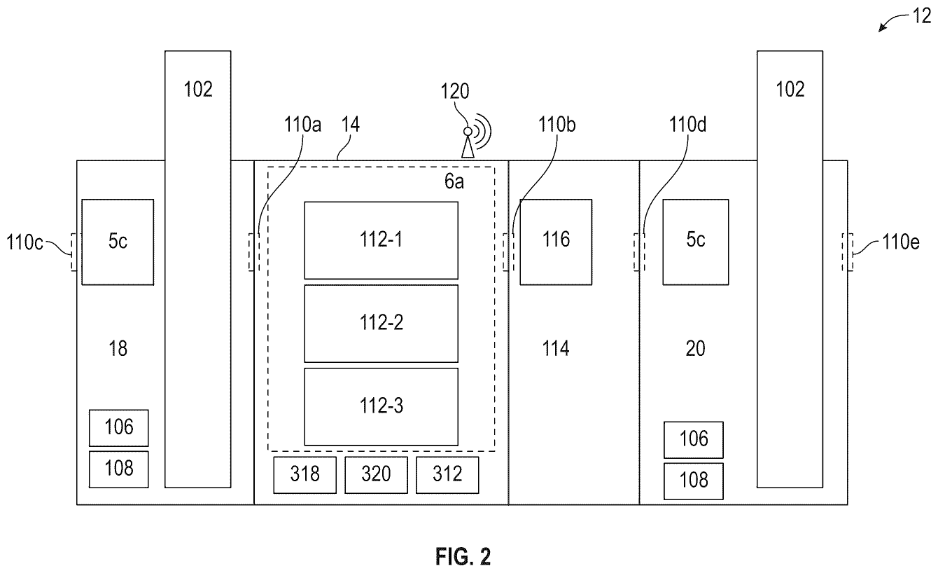

FIG. 2 illustrates a patient care unit 12 with connected infusion device modules 18, 20 configured to deliver a fluid, a main frame infusion controller 14, and a modular infusion control device 114. Each of infusion device modules 18, 20 may include a memory storing instructions and a processor configured to execute the instructions to cause the module to perform at least partially some of the steps in methods as disclosed herein (e.g., memory and processor). While two infusion device modules 18, 20 are described herein, it is understood that one or more than two infusion device modules may be used in patient care unit 12.

Each infusion device module 18, 20 may be electronically coupled to the main frame infusion controller 14 via a respective connector port 110 associated with the main frame. The term “port” as used herein includes a physical interconnect on an electronic device that interfaces with a corresponding physical interconnect of another electronic device to electronically connect with, and communicate with, the other electronic device. Each connector port 110a-e may include electrical terminals so that a coupled infusion device module 18, 20 may transmit and receive information to and from a main frame 14. In some implementations, the infusion device module may also receive power from main frame 14 through a connector port 110. In this regard, the connector port may be a connector port directly on a side of the main frame so that the module electrically connects directly to the connector port when it is mounted to the main frame. Or, the connector port may be a connector port on a side of another module such that the first medication delivery module is electronically coupled to the main frame via the other module. For example, module 20 is electrically coupled to main frame infusion controller 14 via ports 110d and 110b.

When an infusion device module is installed, the main frame infusion controller 14 may receive an indication that the module was electronically coupled to the main frame via a connector port associated with the main frame. The main frame may then dynamically display, on the display screen of the main frame in response to receiving the indication, a graphical channel indicator 112 representative of the module. According to some implementations, each channel indicator 112 is displayed oriented toward the connector port and the medication delivery module corresponding to the indication.

According to various implementations, the illustrated infusion control module 114 is provided that can also couple via a port 110 to the main frame infusion controller 14. As will be described further, the infusion control module 114 includes a processor and a memory, and is configured to electrically couple to the main frame infusion controller 14 and take over control of the patient care device (e.g., the main frame 14 and/or coupled infusion device modules). In some implementations, the control module 114 may have the same size and profile of other infusion device modules 18, 20. In some implementations, the control module 114 is just large enough to include its processing circuitry and memory, and connection ports 110, and may be a different size or shape than the other connected modules, still enabling physical and electrical interconnectivity between the functional modules, control module and main frame devices.

The infusion control module 114, when authorized, may have direct control over connected medical devices, including main frame infusion controller 14 and/or operably connected infusion device modules 18, 20 to assist the clinician in providing a more centralized control and management of the devices. For the purposes of this disclosure, the interactivity between the infusion control module and the patient care unit 12 are described with respect to main frame infusion controller 14 and/or operably connected infusion device modules 18, 20. It is understood that, in some implementations, main frame infusion controller 14 and/or operably connected infusion device modules 18, 20 may be integrated such as to function as a single PCU device.

The infusion control module 114 provides a modular interface system by which various sensors used in a medical environment may be connected to the control module 114 in a generic way to facilitate control over the other connected medical devices 14, 18, 20. For example, connected sensors may include one or more of a heart rate monitor, an oxygen sensor, and an intravenous (IV) flow rate monitor, all of which may be connected to the infusion control module 114 to facilitate, in addition to input by the clinician, centralized control of one or more infusion devices. Connectivity to the various sensors may also be by way of commandeering another device's connected sensors and programmatic sensor control as part of the control module's 114 control of connected devices.

The infusion control module 114 may further connect to a server and/or cloud-based system for further data input, data coordination, and reporting. Examples of cloud-based systems include a cloud-based drug information database (or formulary), and a hospital network 10 that includes electronic medical record (EMR) system 30 or database 37.

In some implementations, the infusion control module 114 includes control software, that provides processing for control algorithms, and connectivity circuitry and/or software to enable closed and semi-closed-loop control capabilities over one or medical devices at a point of use. In some implementations, the infusion control module 114 provides an external interface, for example a user interface 116, for interaction between infusion device modules 18, 20 and one or more different physiological or biometric sensors, and for providing input parameters that may be used to control the titration of IV infusions of medications to a patient. In some implementations, infusion control module 114, when connected to the main frame infusion control device 14, may control operation of interface elements 6a-c (e.g., physical and/or virtual) for the purpose of interaction between infusion device modules and sensors and for providing the input parameters.

In some implementations, the infusion control module 114 provides control for the IV infusion of the medication by an infusion pump (e.g., of an infusion device module) according to the feedback provided by physiological/biometric sensors used to monitor the therapy being performed. In situations where the IV medication is not being delivered as a result of abnormal conditions such as the IV line being occluded, leaking, or not being connected to the patient, the sensors may indicate that the patient is not responding to the therapy. The infusion control module 114 may detect such activity and provide feedback (e.g., a notification or alarm) to the clinician to alert the clinician of a possible fault condition that should be addressed, in some instances before a critical situation occurs.

The infusion control module 114 can incorporate control software (including, e.g., one or more algorithms in the unit) that can be tailored to specific or general medical treatments. The infusion control module 114 may receive infusion status information from the main frame device 14 or the infusion device modules 18, 20. The infusion status information may include, for example, an identification of the medication, a flow rate of an administration of medication to a patient by the infusion device, a VTBI (volume to be infused), delivery duration, upstream or downstream pressure, and the like. While an infusion pump and/or module may have its own safety system, the infusion control module 114 may be preprogrammed to determine safety events such as events specific to a particular procedure. The infusion control module 114 may determine, based on sensor data from the connected sensor(s) and infusion status information, that a safety event is likely to occur within a predetermined period of time (e.g., programmed into the ICM or determined by AI based on training models for the procedure being undertaken).

A closed-loop control system as described herein generally refers to a system that does not rely on external manual inputs to deliver a therapy. Once configured, the closed-loop system can autonomously provide a therapy, receive feedback from one or more sensors and, based on the feedback, automatically adjust the therapy as needed. For example, the infusion control module 114 may determine, during an administration of a medication, an expected trend in a physiological property during a predetermined time period based on sensor data for a prior period of time, a dose of the medication provided to the patient, and the one or more physical parameters of the patient. The infusion control module 114 may then cause the infusion device to adjust the dose of the medication to cause the physiological property to follow an expected trend within a predetermined time period.

Having the infusion control module 114 as a separate modular unit that electrically and removably connects as module to the PCU 12 has several advantages. For example, the advancement of sensors (e.g., biometric sensor(s)) connected to or controlled by the infusion control module 114 may be much more rapid than the development of the PCU 12 or its other modules, and thus the control system may accommodate these changes more quickly. It may be desirable to update control algorithms to address changes in treatment methods, available medications, and patient physiology. For this reason, it may be desirable to have the algorithm reside in a component different than the pump system, in order to accommodate more frequent changes. Moreover, machine learning and artificial intelligence may account for patient variations related to physiological properties such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone, but may reside on a server 30 or other cloud-based system accessible from the infusion control module 114.

Additionally, the infusion control module 114 of the subject technology may be adaptable to a variety of pump systems and sensor inputs (e.g., using universal connector ports 112). The infusion control module 114 may further be configured to add wireless, Bluetooth and LAN connections to pump systems that do not currently have connector ports 110. For example, the FIG. 1C shows the infusion control module 114 having a wireless connection 120 for communication with a network system 40 at the hospital. Adding such communications to the PCU 12 may enable other capabilities such as remote monitoring and control of the infusion pumps, and facilitating access to an EMR. Accordingly, by integrating the electrical and processing components separate from the main frame infusion control device 14, the subject technology facilitates integration of additional capabilities without needing to modify the main frame's housing and electronics. Separating the physiological sensing and control systems from the infusion pump system may further provide for a more streamlined regulatory approval process.

According to various implementations, the infusion control module 114 facilitates having control software separate from the embedded firmware of the pumps and sensors, which can facilitate a scalable and rapidly configured system to provide closed-loop control of medical treatments. Moreover, the infusion control module 114 can be configured to operate with a multitude of sensors by way of electrical connectors and/or wireless communication. The infusion control module 114 may include one or more microprocessors and algorithms to provide signal conditioning and/or conversion of the sensor signals to the appropriate physiological properties for the connected medical device. The parameters may then be used in control algorithms to provide control to, for example, the main frame infusion controller 14 and/or an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

A user interface associated with the infusion control module 114 may include a display module or a touch-sensitive display module configured to provide a user interface for display of information pertaining to patient physiological status, as well as system control status. In some implementations, the infusion control module 114 includes circuitry that provides display information to an external display device (e.g., a terminal 32). The external display may, for example, display various vital statistics (e.g., electrocardiography (ECG), oxygen saturation level (SpO2)) of a patient in a surgery room such that the vital statistics are visible to the clinicians (e.g., all the clinicians) in the room. In some implementations, a display on the infusion control module 114 may be mirrored via an associated external display to provide information to devices connected to the infusion control module 114 and/or clinicians involved in the treatment.

According to various implementations, the software of infusion control module 114 may store protocol information (e.g., information about processes and steps associated with therapies and patients), making the information readily accessible in the infusion control module 114. In some implementations, the software provides information to the user interface, allowing the system to more efficiently (e.g., using fewer device resources such as memory, power, processing cycles, user interface area, and the like) present and navigate through pertinent information (e.g., using touch inputs on a touch screen). According to variously implementations, the software guides the user to the order of steps and information needed at the appropriate time thereby avoiding expenditures of resources on information that is untimely or irrelevant to the currently detected condition.

In some implementations, the infusion control module 114 may access additional resources relating to medication information and dosage calculations from an internal or remote storage (e.g., from a drug information database). In some implementations, the infusion control module 114 can cause a user interface, and/or other operably connected user devices to display information pertaining to a particular medication being administered.

According to various implementations, when coupled to the main frame infusion controller 14, the control module 114 instructs the main frame infusion controller to switch from a default mode to a remote control mode. In this regard, responsive to the infusion control module being coupled to the main frame infusion controller, the user of the PCU 12 may be prompted (e.g., via display 6a) to confirm that the control module 114 may take over control of the PCU. In some implementations, whether the user may provide the confirmation may depend on an authorization level of the user. The authorization level may be determined automatically by the infusion control module 114 based accessing authorization information from the main frame 14 (e.g., obtained based on logging into the main frame by way of scanning a badge).

When in the control mode, operating parameters associated with an infusion may be controlled by the infusion control module 114, while fundamental pumping systems developed for the pump (e.g., calculating and/or controlling volume per mechanical revolution) may continue to be managed by the pump. For example, the infusion control module 114 may take over control of titration rate, starting and stopping of an infusion, and may consume alarms provided by a module 18, 20 for its own use. However, background functionality such as maintaining a programmed flow rate (e.g., once programmed) may be left to the pumping systems of the module 18, 20, thereby leaving the module 18, 20 to function as a zombie module. Under closed-loop control, the infusion control module 114 may monitor and instruct changes to operation parameters in real time to maintain the patient in a designated state (e.g., by way of keeping a physiological sensor measurements within a target zone). Under a semi-closed-loop control, the infusion control module 114 may request confirmation of parameter changes from a user (e.g., via display 6a). In some implementations, infusion control module 114 merely monitors and provides reporting of monitored conditions, alarms, and measurements to a display 6a or external system(s).

According to various implementations, the infusion control module 114 may be disconnected (e.g., physically removed) from a first main frame infusion controller 14a and reconnected to a second main frame infusion controller 14b. In this regard, the infusion control module 114 may locally store in its memory infusion data, patient information, and other medical information so that, when it is connected to the new main frame, an infusion therapy may be continued using the new main frame infusion controller and its connected modules, or a new infusion initiated on that device may make therapy based decisions based on historical data obtained at the first device.

When moved to a new second infusion system, the infusion control module 114 may transfer data obtained by the first system to the second system. The module may first perform a handshake with the new system and/or a data check and/or firmware check. The infusion control module 114 may then determine what features are available on the new system for continuing a therapy previously provided by the infusion control module using a different PCU/main frame infusion controller. For example, the infusion control module 114 may query the new main frame 14b to determine what infusion channels are active and what medications have been loaded for administration by the channels. The infusion control module 114 may determine that one or more medications loaded for administration in the new PCU is compatible with an infusion therapy previously programmed into the infusion control module 114 while the module was coupled to the first main frame infusion controller 14a.

In some implementations, the infusion control module 114 may map medication therapies programmed into the infusion control module 114 to infusion device modules 18, 20 coupled to the new main frame infusion controller 14b. In some implementations, the mapping may be done based on a comparison of operating parameters programmed into the new second main frame infusion controller 14 with parameters programmed into the infusion control module 114. The parameters may include, for example, identification of a medication or medication type, a flow rate of an administration of medication to a patient by the infusion device, a VTBI (volume to be infused), delivery duration, upstream or downstream pressure, and the like.

When available channels for continuing an infusion therapy are identified, the infusion control module 114 may prompt (e.g., via display 6a) a user to select at least one of the channels or available medications to be delivered according to the therapy. The user may then select the medication to use in the therapy. In some implementations, the infusion control module 114 may transmit operating parameter values and/or pharmacokinetic information to the new main frame 14b for use in the delivering the therapy.

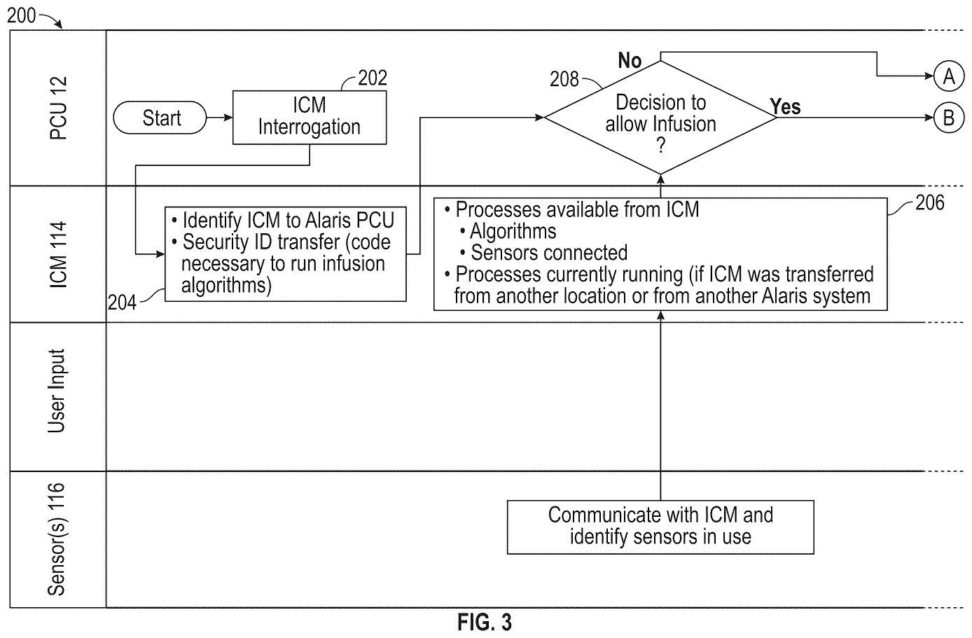

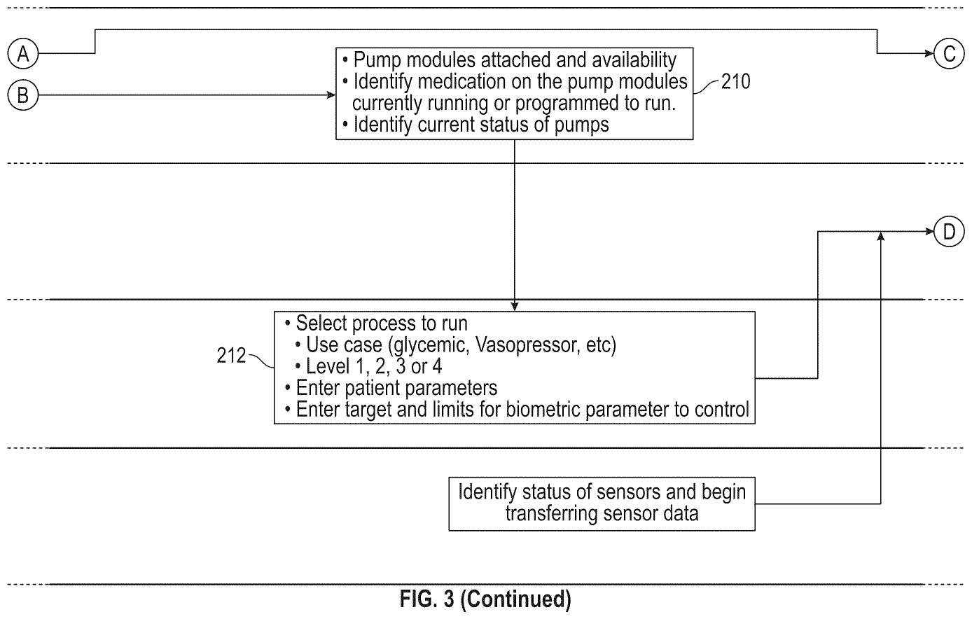

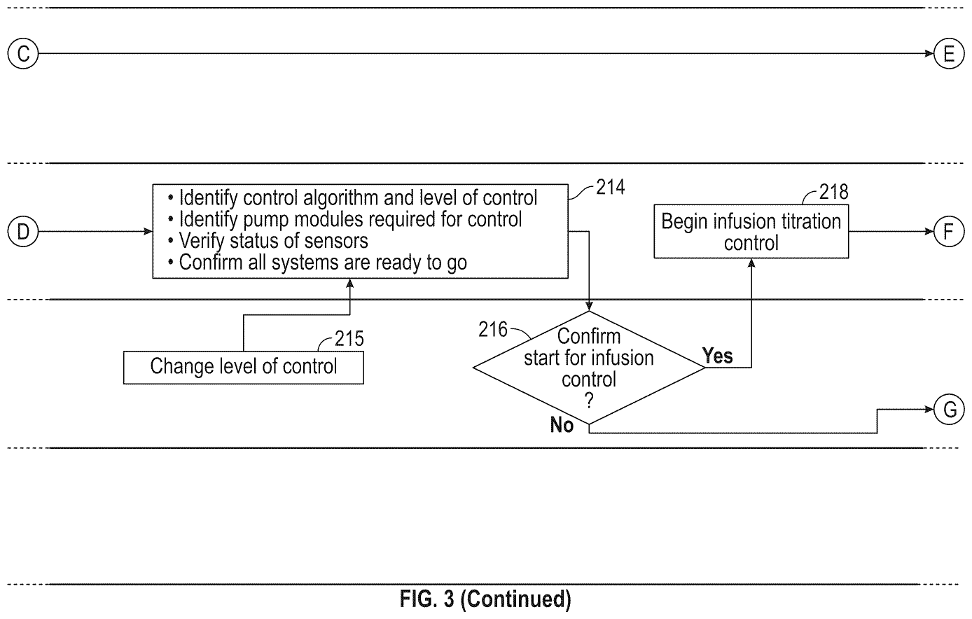

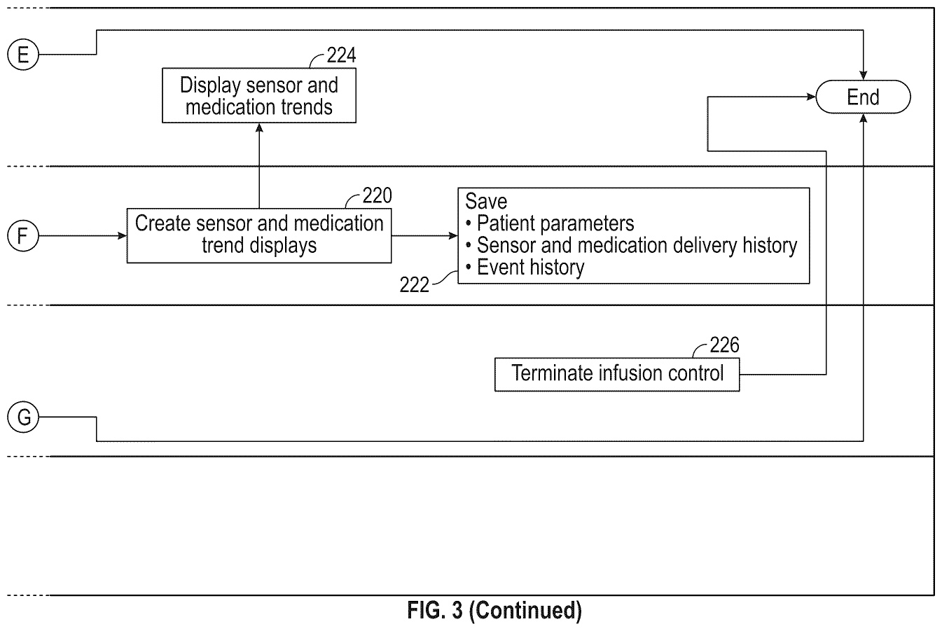

FIG. 3 depicts an example flow diagram 200 for controlling an infusion device using a modular infusion control module, according to aspects of the subject technology. For explanatory purposes, the various blocks of example process flow 200 are described herein with reference to FIGS. 1A, 1B, and 2, and the associated components and/or processes described herein.

As described previously, an infusion control module 114 is electronically coupled to a patient care unit (PCU 12) (e.g., to a main frame infusion controller 14 and/or one or more functional modules thereof), initiating the example process 200. In the depicted example, the infusion control module 114 interrogates the PCU 12 to determine whether the device 14 is compatible with the infusion control module 114 (202). The determination may be based on communication of one or more messages between the ICM 114 and the PCU 12. In some implementations, the determination may be based on physical or virtual connection status between the ICM 114 and the PCU 12.

The infusion control module 114 identifies itself to the PCU 12, and provides a security identifier or code to the PCU 12 via one or more messages (204). Meanwhile, the infusion control module 114 may determine what sensors are connected to the infusion control module 114 or that where previously connected on another system prior to being connected to the present PCU 12 (206). In some implementations, the sensor information may be provided by the PCU 12 (e.g., based on sensors connected to the PCU or a coupled module). The PCU 12 receives the indication of identity and security code and, based thereupon, determines whether access should be granted to the infusion control module 114 (208). For example, if the identifier for the ICM 114 is not included in a list of allowed ICMs, the determination may be negative. As another example, if the security code provided by the ICM 114 is unknown, expired, or invalid, the determination may be negative. If the PCU 12 does not accept the received identity and/or code(s) then the process ends.

On acceptance of the identity and/or code(s), the PCU 12 may provide the infusion control module 114 with information pertaining to its operating systems (210). For example, the PCU 12 may identify infusion device modules 18, 20 currently coupled to the main frame infusion controller 14 and/or which of the modules are available for use, and/or which modules are programmed with operating parameters and the values of those parameters. The PCU 12 may identify one or more medications associated with (e.g., loaded on) pump modules 18, 20 currently running or programmed to run, and may identify a current status of each pump module (e.g., whether the pump module is actively infusing the medication). In some implementations, the foregoing information may be provided by the PCU 12 in response to a query for the information from the infusion control module 114.

As previously described, the infusion control module 114 may assume control of (e.g., commandeer) a display screen 6a and/or input interface controls 6b, 6c of the PCU 12. In this regard, the infusion control module 114 may prompt a user to select a process to run or to set up a therapy. For example, the infusion control module 114 may prompt a user to select a glycemic therapy, vasopressor therapy, etc. The user may then select the appropriate therapy and enter patient parameters, sensor targets and/or limits, and other operating parameters for the therapy using the user interface controls (212). For example, the user may select an anesthesia therapy, a medication such as propofol, an amount to be administered, a bi-spectral index (BIS) sensor, and a predetermined target for measurements of the BIS sensor that corresponds to a depth of anesthesia.

In some implementations, the PCU 12 (or module to be controlled) identifies a control algorithm and/or a level of control of the infusion control module 114 and/or the modules 18, 20 required for the control (214). The level may correspond to required confirmation needed for the ICM 114 to take an automatic action. At a first level, for example, the ICM 114 may simply recommend an adjustment via the PCU 12 during an infusion. At a second level the ICM 114 may directly control a module without any user input during an infusion. Sensor input may be received via the infusion control module 114 or directly by the PCU 12. The PCU 12 may verify the status of the sensors and confirm that all systems are valid, operational, and ready to hand off to the infusion control module 114 for direct control by the infusion control module 114. The user may adjust the level of control over the PCU 12 by the infusion control module 114 by way of providing the appropriate input at user interface 6a, 6b, 6c (215).

The infusion control module 114 may then request a user to confirm that the infusion control module 114 may start to control the PCU 12 (216) (or module connected thereto). The user may cancel or, on confirming, the infusion control module 114 may begin control of the PCU 12 (or module connected thereto). In this regard, the infusion control module 114 may begin titration control. In some implementations, the infusion control module 114 begins monitoring sensor measurements and titrating the medication based on the measurements. The infusion control module 114 may generate sensor and medication trend data displays for display on a display screen (220) and log the same data (222). The infusion control module 114 may cause a user interface to be displayed on the PCU 12 with the generated trend displays and other real time monitoring data (224). At any time, the user may terminate control over the PCU 12 by the infusion control module 114 (226), for example, by providing the appropriate input at user interface 6a, 6b, 6c.

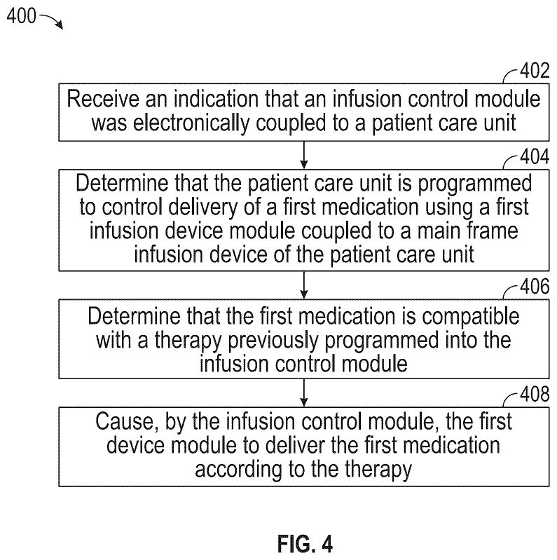

FIG. 4 depicts an example process 400 for controlling an infusion device using a modular infusion control module, according to aspects of the subject technology. For explanatory purposes, the various blocks of example process 400 are described herein with reference to FIGS. 1A, 1B, 2, and 3, and the associated components and/or processes described herein. The one or more of the blocks of process 400 may be implemented, for example, by one or more computing devices including, for example, infusion control module 114, PCU 12, module 18, 20, server 30, or a client computing device 32. In some implementations, one or more of the blocks may be implemented based on one or more machine learning algorithms. In some implementations, one or more of the blocks may be implemented apart from other blocks, and by one or more different processors or devices. Further, for explanatory purposes, to the extent that the blocks of example process 400 are described as occurring in serial, or linearly, in some implementations, multiple blocks of example process 400 may occur in parallel (e.g., blocks 402 and 404 may occur in parallel). In addition, the blocks of example process 400 need not be performed in the order shown and/or one or more of the blocks of example process 400 need not be performed.

According to various implementations, an infusion control module 114 receives an indication that the infusion control module 114 was electronically coupled to a patient care unit (PCU) 12 (402). According to various implementations, a main frame infusion controller of the PCU is configured to control one or more infusion device modules 18, 20 coupled to the PCU. The infusion control module 114 may become electronically coupled to the PCU 12 by way of a connector port 110 of the control module 114 physically interfacing and interconnecting with a connector port 110 of the main frame infusion controller 14. The infusion control module 114 may also become coupled by way of the port of the control module 114 interfacing with a connector port of a module 18, 20 that is, in turn, coupled to the main frame infusion controller 14. In this regard, the module 18, 20 may include pass-through circuitry that allows the main frame infusion controller 14 and the infusion control module 114 to be perceived as being coupled irrespective of the intervening module(s). In some implementations, a second physical connector port 110d may removably couple the infusion control module 114 to an infusion device module (e.g., to infusion device module 20, as depicted in FIG. 2).

Responsive to the infusion control module 114 being coupled to the PCU, the control module may prompt a user to confirm switching the main frame infusion controller 114 from a default mode in which control of the PCU (e.g., main frame infusion controller 14 and/or connected infusion device modules) is controlled by the PCU to a remote control mode in which control of the PCU is controlled by the infusion control module when electronically coupled to the PCU. Responsive to receiving the user confirmation, the control module and/or the PCU are switched to the remote control mode. While in the remote control mode, the control module 114 may control certain functions of the PCU such as a start time and a stop time and a titration rate of a medication being delivered according to a therapy selected at the control module 114. According to various implementations, the PCU may maintain control of fundamental functions such as maintaining a programmed flow rate of a medication (which, e.g., can be adjusted by the control module 114 in real time).

The infusion control module 114 determines that the PCU is programmed to control delivery of a respective medication using an infusion device module 18, 20 coupled to the main frame infusion controller (404). As described previously, the infusion control module 114 may determine capabilities of the PCU 12, including identifying functional modules connected to the PCU (e.g., to the main frame 14), whether those modules include infusion pumps, the type of pump, the medication loaded for administration by the pump, and/or operating parameters for configuring the pump, and the like. The control module 114 may determine that the main frame infusion controller 14 is programmed to control delivery of two or more medications using two or more infusion device modules 18, 20.

The infusion control module 114 determines that the medication currently associated with the PCU is compatible with a therapy previously programmed into the infusion control module (406). In this regard, the control module may be pre-programmed to control a specific therapy (e.g., anesthesia). The pre-programming may be by way of being previously connected to another PCU 12, or the therapy may be selected when the control module was electronically coupled to the current PCU 12. For example, on being electronically coupled and performing initial configuration set up actions such as those described with respect to FIG. 2, or as part of the configuration set up actions, the control module 12 may prompt the user to select a therapy to be monitored and/or controlled by the control module 114. For example, the therapy may be an anesthesia, or may be glycemic control.

The infusion control module 114 may cause the PCU 12 to prompt a user to select or confirm use of the medication loaded by the PCU. For example, the control module 114 may determine that two or more modules 18, 20 are active on the PCU and each available to deliver a medication, and may list the modules on the display 6a for selection by the user. The user may then select one or more of the listed medications to be delivered according to the therapy provided by the control module 114.

In some implementations, the infusion control module 114 may determine that a medication loaded by the PCU is compatible with the therapy previously programmed into the infusion control module while the infusion control module was coupled to a different main frame device. For example, the infusion control module 114 may be programmed for an anesthesia therapy and previously programmed to use propofol for the therapy. When the control module 114 is coupled to the PCU 12, the module may determine that remifentanil is available from the PCU 12. The module 114 may then prompt the user for the user to confirm the use of remifentanil in place of propofol.

In some implementations, the infusion control module 114 may transmit, from the infusion control module to the main frame infusion controller, parameters and pharmacokinetic information programmed into the infusion control module while the infusion control module was coupled to the different main frame device. In this manner, the control module 114 may be used to transfer an entire therapy regime for a patient from a first PCU administering medication for the patient to a second PCU, and the therapy continued for the patient on the second PCU. The second PCU may use the transferred parameters and pharmacokinetic information with the same type of medication, or may use and/or convert for use the parameters and/or pharmacokinetic information with a similar type of medication (e.g., remifentanil instead of propofol or vice versa). In some implementations, the control module 114 maps (e.g., based on parameters and/or medication types programmed into the main frame infusion controller) medication therapies programmed into the infusion control module when electronically coupled to the previous PCU to the current PCU (or, e.g., infusion device modules coupled to the main frame of the current PCU).

In the depicted example, the infusion control module 114 causes, while electronically coupled to the PCU, the infusion device module to deliver the medication according to the therapy (408). As described previously, the control module 114 may, for example, set operating parameters of the main frame infusion controller 14 or of the infusion device module 18, 20, and the PCU may operate as if the parameters were received directly without the control module. However, the control module 14, being coupled to the PCU, may continuously monitory the PCU and its modules, and make adjustments to parameters (e.g., flow rate, VTBI, bolus amounts, etc.) in real time to ensure that an administration of a medication maintains a patient in a desired state (e.g., based on physiological sensor measurements).

According to various implementations, an infusion device module (e.g., module 18 or 20) coupled to the main frame infusion controller 14 may be caused by the control module 114 to deliver the medication by way of sending one or more signals from the infusion control module 114 to the main frame infusion controller via a physical interconnect (e.g., port 110b in FIG. 2). In some implementations, the infusion device module may be caused by the control module 114 to deliver the medication by way of sending the one or more signals from the infusion control module to the infusion device module (e.g., module 20) via a second physical interconnect (e.g., port 110d in FIG. 2). In some implementations, when coupled to the PCU, the control module 114 may obtain, from a server 30, one or more parameter limits (e.g., guardrails) associated with the medication and/or the patient. The control module 114 may then monitor delivery of the medication for a contravention of the one or more parameter limits and, responsive to a contravention, instruct the PCU to adjust a parameter associated with the administration of the medication to the patient (e.g., adjust the flow rate of the medication).

In some implementations, for example during a setup or initiation of the control module (e.g., FIG. 2), the control module 114 determines that the PCU is programmed to receive sensor data from a sensor operably coupled to the PCU. Biometric sensors used with the control module 114 and/or with a PCU may include an HR monitor, an oxygen sensor, and an intravenous (IV) flow rate monitor, all of which may facilitate, in addition to input by the clinician, centralized control of one or more infusion pumps provided by the PCU. In some implementations, the sensors may also include a bi-spectral index (BIS) sensor to assess the depth of anesthesia during an operation.

The control module 114 may then determine that the sensor is compatible with the therapy and prompt a user to confirm use of the sensor in connection with delivery of the medication according to the therapy. The control module 114 may receive a user confirmation that the sensor is used in connection with the delivery, receive sensor data from the sensor, and then adjust a parameter of the infusion device module during the therapy based on the sensor data.

Many of the above-described example process 400 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

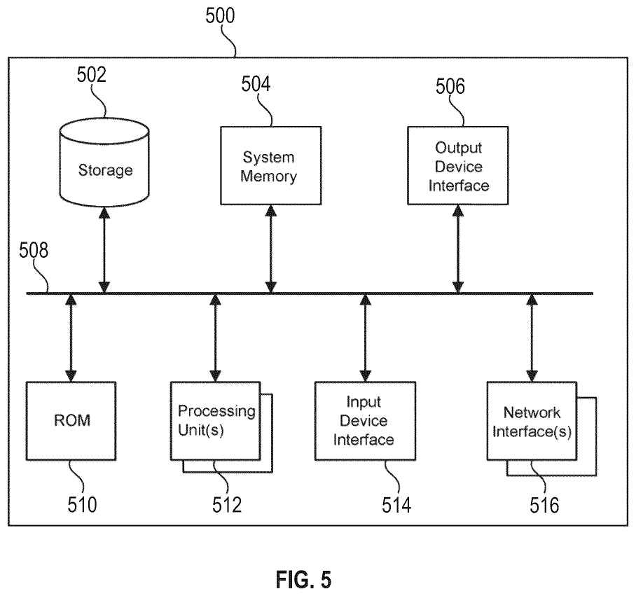

FIG. 5 is a conceptual diagram illustrating an example electronic system 500 for controlling an infusion device using a modular infusion control module, according to aspects of the subject technology. Electronic system 500 may be a computing device for execution of software associated with one or more portions or steps of processes 200 and 400, or components and methods provided by FIGS. 1-3, including but not limited to computing hardware within the PCU 12, main frame infusion controller 14, control module 114, functional modules 16, 18, 20, 22, server 30, terminal 32, and/or any computing devices or associated modules or terminals disclosed herein. Electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and methods previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as, random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.
Clause 1. An infusion control module, comprising: a non-transitory machine readable storage medium comprising instructions stored thereon; and one or more processors configured by the instructions to: receive an indication that the infusion control module was electronically coupled to a main frame infusion controller, the main frame infusion controller being configured to control one or more infusion device modules coupled to the main frame infusion controller; determine, by the infusion control module, that the main frame infusion controller is programmed to control delivery of a medication using an infusion device module coupled to the main frame infusion controller; determine, by the infusion control module, that the medication is compatible with a therapy previously programmed into the infusion control module; and cause, by the infusion control module while electronically coupled to the main frame infusion controller, the infusion device module to deliver the medication according to the therapy.
Clause 2. The infusion control module of Clause 1, wherein the main frame infusion controller is programmed to deliver two or more medications using two or more infusion device modules, the one or more processors being further configured by the instructions to: prompt, via the infusion control module, a user to select at least one of the two or more medications to be delivered according to the therapy; and receive, based on the prompt, a selection of the medication.
Clause 3. The infusion control module of Clause 1 or Clause 2, wherein: determining that the medication is compatible with the therapy previously programmed into the infusion control module comprises determining that the medication is compatible with the therapy previously programmed into the infusion control module while the infusion control module was coupled to a different main frame device.
Clause 4. The infusion control module of Clause 3, the one or more processors being further configured by the instructions to: map, based on parameters programmed into the main frame infusion controller, a plurality of medication therapies programmed into the infusion control module when electronically coupled to the different main frame device to a plurality of infusion device modules coupled to the main frame infusion controller, the parameters comprising one or more medication types.
Clause 5. The infusion control module of Clause 3, the one or more processors being further configured by the instructions to: transmit, from the infusion control module to the main frame infusion controller, parameters and pharmacokinetic information programmed into the infusion control module while the infusion control module was coupled to the different main frame device.
Clause 6. The infusion control module of any one of Clauses 1 through 5, the one or more processors being further configured by the instructions to: receive, by the infusion control module, infusion information pertaining to a delivery of the medication to a patient; generate, by the infusion control module, a user interface; and cause, by the infusion control module, a display device of the main frame infusion controller to display the user interface with infusion control parameters and information pertaining to the delivery of the medication based on the received infusion information.
Clause 7. The infusion control module of any one of Clauses 1 through 6, the one or more processors being further configured by the instructions to: prompt, responsive to the infusion control module being coupled to the main frame infusion controller, a user to confirm switching the main frame infusion controller from a default mode in which control of the main frame infusion controller and the infusion device module are controlled by the main frame infusion controller to a remote control mode in which control of the main frame infusion controller and the infusion device module are controlled by the infusion control module when electronically coupled to the main frame infusion controller; switch, responsive to receiving the user confirmation, the main frame infusion controller to the remote control mode; and control, by the infusion control module, while in the remote control mode, a start time and a stop time and a titration rate of the medication being delivered according to the therapy, wherein the main frame infusion controller maintains control of a flow rate of the medication during the remote control mode.
Clause 8. The infusion control module of Clause 7, the one or more processors being further configured by the instructions to: obtain, from a server, by the infusion control module, responsive to the infusion control module being coupled to the main frame infusion controller, one or more parameter limits associated with the medication; monitor delivery of the medication for a contravention of the one or more parameter limits; and responsive to the contravention, instructing, by the infusion control module, the main frame infusion controller to adjust the flow rate of the medication.
Clause 9. The infusion control module of any one of Clauses 1 through 8, the one or more processors being further configured by the instructions to: determine, by the infusion control module, that the main frame infusion controller is programmed to receive sensor data from a sensor operably coupled to the main frame infusion controller; determine that the sensor is compatible with the therapy; prompt a user to confirm use of the sensor in connection with delivery of the medication by the infusion device module according to the therapy; receive a confirmation that the sensor is used in connection with the delivery; receive sensor data from the sensor; and adjust, by the infusion control module, a parameter of the infusion device module during the therapy based on the sensor data.
Clause 10. The infusion control module of any one of Clauses 1 through 9, the one or more processors being further configured by the instructions to: a first physical interconnect configured to physically and removably couple to the main frame infusion controller, wherein the indication that the infusion control module was electronically coupled to the main frame infusion controller is received via the first physical interconnect.
Clause 11. The infusion control module of Clause 10, wherein the infusion device module being caused to deliver the medication is by way of one or more signals being sent from the infusion control module to the main frame infusion controller via the first physical interconnect.
Clause 12. The infusion control module of Clause 10, the one or more processors being further configured by the instructions to: a second physical interconnect configured to physically and removably couple to the infusion device module when the first physical interconnect is physically coupled to the main frame infusion controller, wherein the infusion device module being caused to deliver the medication is by way of one or more signals being sent from the infusion control module to the infusion device module via the second physical interconnect.
Clause 13. A machine-implemented method, comprising: receiving, by an infusion control module, an indication that the infusion control module was electronically coupled to a main frame infusion controller, the main frame infusion controller being configured to control one or more infusion device modules coupled to the main frame infusion controller; determining, by the infusion control module, that the main frame infusion controller is programmed to deliver a medication using a infusion device module coupled to the main frame infusion controller; determining, by the infusion control module, that the medication is compatible with a therapy previously programmed into the infusion control module; and causing, by the infusion control module while electronically coupled to the main frame infusion controller, the infusion device module to deliver the medication according to the therapy.
Clause 14. The machine-implemented method of Clause 13, wherein the main frame infusion controller is programmed to deliver two or more medications using two or more infusion device modules, the method further comprising: prompting, via the infusion control module, a user to select at least one of the two or more medications to be delivered according to the therapy; and receiving, based on the prompt, a selection of the medication.
Clause 15. The machine-implemented method of Clause 13 or Clause 14, wherein: determining that the medication is compatible with the therapy previously programmed into the infusion control module comprises determining that the medication is compatible with the therapy previously programmed into the infusion control module while the infusion control module was coupled to a different main frame infusion controller.
Clause 16. The machine-implemented method of Clause 15, the method further comprising: mapping a plurality of medication therapies programmed into the infusion control module when coupled to the different main frame infusion controller to a plurality of infusion device modules coupled to the main frame infusion controller based on parameters programmed into the main frame infusion controller, the parameters comprising one or more medication types.
Clause 17. The machine-implemented method of any one of Clauses 13 through 16, the method further comprising: prompting, responsive to the infusion control module being coupled to the main frame infusion controller, a user to confirm switching the main frame infusion controller from a default mode in which control of the main frame infusion controller and the infusion device module are controlled by the main frame infusion controller to a remote control mode in which control of the main frame infusion controller and the infusion device module are controlled by the infusion control module; switching, responsive to receiving the user confirmation, the main frame infusion controller to the remote control mode; and controlling, by the infusion control module, while in the remote control mode, a start time and a stop time and a titration rate of the medication being delivered according to the therapy, wherein the main frame infusion controller maintains control of a flow rate of the medication during the remote control mode.
Clause 18. The infusion control module of any one of Clauses 13 through 17, comprising: receiving the indication that the infusion control module was electronically coupled to the main frame infusion controller via a first physical interconnect of the infusion control module, the first physical interconnect removably coupling the infusion control module to the main frame infusion controller.
Clause 19. The infusion control module of Clause 18, comprising: causing the infusion device module being to deliver the medication by way of sending one or more signals from the infusion control module to the main frame infusion controller via the first physical interconnect, or by way of by way of sending one or more signals from the infusion control module to the infusion device module via a second physical interconnect of the infusion control module, the second physical interconnect removably coupling the infusion control module to the infusion device module.
Clause 20. A non-transitory machine-readable storage medium having instructions stored thereon that, when executed, cause an infusion control module to perform a method according to any one of Clauses 13 through 19.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. An infusion control module, comprising:
a non-transitory machine readable storage medium comprising instructions stored thereon; and
one or more processors configured by the instructions to:
receive an indication that the infusion control module was electronically coupled to a main frame infusion controller, the main frame infusion controller being configured to control one or more infusion device modules coupled to the main frame infusion controller;
determine, by the infusion control module, that the main frame infusion controller is programmed to control delivery of a medication using an infusion device module coupled to the main frame infusion controller;
determine, by the infusion control module, that the medication is compatible with a therapy previously programmed into the infusion control module; and
cause, by the infusion control module while electronically coupled to the main frame infusion controller, the infusion device module to deliver the medication according to the therapy.

2. The infusion control module of claim 1, wherein the main frame infusion controller is programmed to deliver two or more medications using two or more infusion device modules, the one or more processors being further configured by the instructions to:
prompt, via the infusion control module, a user to select at least one of the two or more medications to be delivered according to the therapy; and
receive, based on the prompt, a selection of the medication.

3. The infusion control module of claim 1, wherein:
determining that the medication is compatible with the therapy previously programmed into the infusion control module comprises determining that the medication is compatible with the therapy previously programmed into the infusion control module while the infusion control module was coupled to a different main frame device.

4. The infusion control module of claim 3, the one or more processors being further configured by the instructions to:
map, based on parameters programmed into the main frame infusion controller, a plurality of medication therapies programmed into the infusion control module when electronically coupled to the different main frame device to a plurality of infusion device modules coupled to the main frame infusion controller, the parameters comprising one or more medication types.

5. The infusion control module of claim 3, the one or more processors being further configured by the instructions to:
transmit, from the infusion control module to the main frame infusion controller, parameters and pharmacokinetic information programmed into the infusion control module while the infusion control module was coupled to the different main frame device.

6. The infusion control module of claim 1, the one or more processors being further configured by the instructions to:
receive, by the infusion control module, infusion information pertaining to a delivery of the medication to a patient;
generate, by the infusion control module, a user interface; and
cause, by the infusion control module, a display device of the main frame infusion controller to display the user interface with infusion control parameters and information pertaining to the delivery of the medication based on the received infusion information.

7. The infusion control module of claim 1, the one or more processors being further configured by the instructions to:
prompt, responsive to the infusion control module being coupled to the main frame infusion controller, a user to confirm switching the main frame infusion controller from a default mode in which control of the main frame infusion controller and the infusion device module are controlled by the main frame infusion controller to a remote control mode in which control of the main frame infusion controller and the infusion device module are controlled by the infusion control module when electronically coupled to the main frame infusion controller;
switch, responsive to receiving the user confirmation, the main frame infusion controller to the remote control mode; and
control, by the infusion control module, while in the remote control mode, a start time and a stop time and a titration rate of the medication being delivered according to the therapy, wherein the main frame infusion controller maintains control of a flow rate of the medication during the remote control mode.

8. The infusion control module of claim 7, the one or more processors being further configured by the instructions to:
obtain, from a server, by the infusion control module, responsive to the infusion control module being coupled to the main frame infusion controller, one or more parameter limits associated with the medication;
monitor delivery of the medication for a contravention of the one or more parameter limits; and
responsive to the contravention, instructing, by the infusion control module, the main frame infusion controller to adjust the flow rate of the medication.

9. The infusion control module of claim 1, the one or more processors being further configured by the instructions to:
determine, by the infusion control module, that the main frame infusion controller is programmed to receive sensor data from a sensor operably coupled to the main frame infusion controller;
determine that the sensor is compatible with the therapy;
prompt a user to confirm use of the sensor in connection with delivery of the medication by the infusion device module according to the therapy;
receive a confirmation that the sensor is used in connection with the delivery;
receive sensor data from the sensor; and
adjust, by the infusion control module, a parameter of the infusion device module during the therapy based on the sensor data.

10. The infusion control module of claim 1, the one or more processors being further configured by the instructions to:
a first physical interconnect configured to physically and removably couple to the main frame infusion controller, wherein the indication that the infusion control module was electronically coupled to the main frame infusion controller is received via the first physical interconnect.

11. The infusion control module of claim 10, wherein the infusion device module being caused to deliver the medication is by way of one or more signals being sent from the infusion control module to the main frame infusion controller via the first physical interconnect.

12. The infusion control module of claim 10, the one or more processors being further configured by the instructions to:
a second physical interconnect configured to physically and removably couple to the infusion device module when the first physical interconnect is physically coupled to the main frame infusion controller, wherein the infusion device module being caused to deliver the medication is by way of one or more signals being sent from the infusion control module to the infusion device module via the second physical interconnect.

13. A machine-implemented method, comprising:
receiving, by an infusion control module, an indication that the infusion control module was electronically coupled to a main frame infusion controller, the main frame infusion controller being configured to control one or more infusion device modules coupled to the main frame infusion controller;
determining, by the infusion control module, that the main frame infusion controller is programmed to deliver a medication using a infusion device module coupled to the main frame infusion controller;
determining, by the infusion control module, that the medication is compatible with a therapy previously programmed into the infusion control module; and
causing, by the infusion control module while electronically coupled to the main frame infusion controller, the infusion device module to deliver the medication according to the therapy.

14. The machine-implemented method of claim 13, wherein the main frame infusion controller is programmed to deliver two or more medications using two or more infusion device modules, the method further comprising:
prompting, via the infusion control module, a user to select at least one of the two or more medications to be delivered according to the therapy; and
receiving, based on the prompt, a selection of the medication.

15. The machine-implemented method of claim 13, wherein:
determining that the medication is compatible with the therapy previously programmed into the infusion control module comprises determining that the medication is compatible with the therapy previously programmed into the infusion control module while the infusion control module was coupled to a different main frame infusion controller.

16. The machine-implemented method of claim 15, the method further comprising:
mapping a plurality of medication therapies programmed into the infusion control module when coupled to the different main frame infusion controller to a plurality of infusion device modules coupled to the main frame infusion controller based on parameters programmed into the main frame infusion controller, the parameters comprising one or more medication types.

17. The machine-implemented method of claim 13, the method further comprising:
prompting, responsive to the infusion control module being coupled to the main frame infusion controller, a user to confirm switching the main frame infusion controller from a default mode in which control of the main frame infusion controller and the infusion device module are controlled by the main frame infusion controller to a remote control mode in which control of the main frame infusion controller and the infusion device module are controlled by the infusion control module;
switching, responsive to receiving the user confirmation, the main frame infusion controller to the remote control mode; and
controlling, by the infusion control module, while in the remote control mode, a start time and a stop time and a titration rate of the medication being delivered according to the therapy, wherein the main frame infusion controller maintains control of a flow rate of the medication during the remote control mode.

18. The infusion control module of claim 13, comprising:
receiving the indication that the infusion control module was electronically coupled to the main frame infusion controller via a first physical interconnect of the infusion control module, the first physical interconnect removably coupling the infusion control module to the main frame infusion controller.

19. The infusion control module of claim 18, comprising:
causing the infusion device module being to deliver the medication by way of sending one or more signals from the infusion control module to the main frame infusion controller via the first physical interconnect, or
by way of by way of sending one or more signals from the infusion control module to the infusion device module via a second physical interconnect of the infusion control module, the second physical interconnect removably coupling the infusion control module to the infusion device module.

20. A non-transitory machine-readable storage medium having instructions stored thereon that, when executed, cause an infusion control module to perform a method according to claim 13.
